(19) United States
(12) Patent Application Publication
(10) Pub. No.: US 2008/0199517 A1
(43) Pub. Date: Aug. 21, 2008

(54) Composition and a process thereof

(75) Inventors: Bhaskaran Sunil, Pune (IN); Vishwaraman Mohan, Pune (IN)

Correspondence Address: OLIFF & BERRIDGE, PLC, P.O. BOX 320850, ALEXANDRIA, VA, 22320-4850, US

(73) Assignee: INDUS BIOTECH PRIVATE LTD, PUNE (IN)

(21) Appl. No.: 12/071,117

(22) Filed: Feb. 15, 2008

Related U.S. Application Data
(60) Provisional application No. 60/906,213, filed on Mar. 12, 2007.

(30) Foreign Application Priority Data
Feb. 19, 2007 (IN) 00333/MUM/2007

(51) Int. Cl. A61K 9/48; A61K 31/7032; A61K 8/60

(52) U.S. Cl. 424/456; 514/26; 424/49

(57) ABSTRACT

The present invention relates to a novel herbal composition for improving exercise physiology factors comprising saponins and sugar derivatives optionally along with pharmaceutically acceptable excipients. It also relates to the process of preparing the composition for improving exercise physiology factors comprising the steps of flaking, defatting, solvent extraction of seeds of trigonella species followed by concentration of the extract to obtain a saponin and sugar derivative. It also relates to the use of the composition for improving exercise physiology factors comprising, enhanced anabolic activity, enhanced muscle building, enhance creatine delivery and reuptake, an increase in testosterone levels and an enhanced immunity.

### FIELD OF THE INVENTION

The present invention relates to the development of a novel composition for improving Exercise Physiology Factors. Also the present invention relates to the preparation of a novel composition derived from Trigonella foenum graecum. This invention also relates to improvement of exercise physiology factors like Anabolic Activity, Immune function, improvement and increasing of creatine uptake and recycle in Muscle cells.

### BACKGROUND OF THE INVENTION

Increasing anabolic activity during exercise is an important goal of exercise physiology. Anabolic activity is characterized by enhanced muscle mass and tissue building abilities and is an outcome of exercise physiology. Anabolic activity is mediated by androgenic hormones particularly testosterone. Testosterone is an important hormone, which plays a significant role in enhancing anabolic activity. Most testosterone produced in the body is in the bound form (app 98%). Plasma testosterone is divided into 3 fractions:

i. The fraction bound to sex hormone binding globulin (SHBG).

ii. The fraction bound to Albumin.

iii. The free testosterone

Only about 2% of plasma testosterone is in the free form and circulates in the body. This free testosterone is responsible for physiological action by binding to nuclear receptors and triggering mRNA responsible for protein/peptide synthesis. Due to the labile bonding between albumin and testosterone the latter easily exchanges with free fraction. Consequently the albumin bound fraction possesses important biological activity. In contrast, testosterone binding to SHBG is very strong, as a result of which the biological activity of this fraction is scarce.

Athletes, sportsmen and body builders, perform exercise to improve body mass composition. Many of these athletes take exogenous testosterone and its derivatives and precursors. However, this tends to increase the total testosterone and free testosterone in serum and urine beyond physiological limit. As a result the metabolites will be beyond the permitted limits leading to WADA (World Anti-Doping Agency) attention and disqualifications.

As a result, there has been always search for ingredients, which can increase Anabolic Activity. This is particularly significant in the areas of natural products. One such product, which is increasing anabolic activity, is Tribulus. terrestris extract. This has been standardized for markers like Saponins and protodiosin. These extracts have been extensively studied for libido improvement and for treatment of impotence.

### PRIOR ART

Viktorov, E, Bozadieva et al have studied the pharmacological effects of protodioscin. Pharmacological, pharmacokinetic, I Viktorov.

They have confirmed that this standardized extract has activity in treating impotence and in improving libido. However, his studies do not report any observed changes in increase in testosterone both free and bound. As this standardized extract has certain libido enhancing activity, this extract is used for enhancing anabolic activity. Many formulations based on Tribulus Terrestris extract are currently available.

V K Neychev and V I Mitev et al have published their study in Journal of Ethno pharmacology Vol 101, issue 1-3, 3 Oct. 2005, 319-323.

They have done a 4-week human study with Tribulus terrestris extract at 10 mg/kg and 20 mg/kg dose. However, they have not found any increase in serum testosterone or androstenedione. Thus the effect of Tribulus terrestris on testosterone is doubtful. Therefore, one is unsure about the anabolic activity of Tribulus terrestris. Zinc and magnesium formulations have become very popular for increasing Anabolic Activity. Zinc and Magnesium are building blocks of many enzymes, which are co-factors for hormone production.

Effects of a Novel-Zinc-Magnesium formulation on hormones and strength published in “official Journal of American Society of Exercise Physiologist Vol. 3, issue No. 4, October 2000.

This is a human study in 27 subjects for 8 weeks. As per this study, there is significant increase in serum total testosterone, significant increase in serum free testosterone and significant increase in insulin like growth factor. This study reports that free testosterone levels were 176 pg/ml in the ZMA (Zinc Magnesium Aspartate) treated groups. Physiological limits approved are 8.69 to 54 pg/ml. whereas this reports 3.3 times the upper physiological limits. This can also show up as higher Metabolite levels in serum and urine and can attract WADA (World Anti Doping Agency) disqualification. WADA only accepts physiological limits for these performance enhancing hormones.

Zinc and Magnesium mediated increase in testosterone can be due to endogenous increase in free testosterone production. Endogenous production is subject to negative feedback control and homeostasis. However, the reported study seems to suggest something contrary to this physiological reality.

Enzymes like aromatse which are responsible for the conversion of the Androgen into estradiol and estrone are well known and currently used as targets for the development of breast cancer drugs. It is observed that the serum testosterone (both total and free) levels are highest in the morning. However, during the day it gets metabolized by Aromatase and serum concentrations decline. There are several compounds developed for Aromatase inhibition. One such compound is procyanidin. B dimmers found in grape seed, pine bark, red wine etc. Thus with the use of procyanidin-B, the testosterone degradation can be arrested and the morning levels of high testosterone concentrations can be maintained in serum.

Canadian Patent No. CA 2537648 A1 of Gardiner, et al makes use of this property of procyanidin B for maintaining the morning levels of testosterone. This helps in increasing anabolic activity. However, this does not help to increase the endogenous production of testosterone either total or free.

Therefore, there is an unmet need for a naturally derived safe product which can be used to improve anabolic activity, which has been tested to provide evidence of testosterone boosting activity and anabolic activity.

Besides, anabolic activity, exercise physiology is also concerned with enhancing immunity.

Mackinnon has reported earlier (Medicine and Science in Sports and Exercise 32 (7) pp 369.376 July 2000) that chronic exercise leads to impairment of immunity. This has been further confirmed by the studies of Bente et al in the British Journal of Sports Medicine 2000, 34 pp 246-251. They have demonstrated that chronic exercise leads to impairment of immunology by reduction in total lymphocytes. Thus, contrary to general perception, strenuous exercise induces immune suppression in the recovery period and may explain the increased risk of infection in athletes. Therefore, there is a need for a composition to boost immunity in chronic and strenuous exercise physiology.

Chronic exercise also demands more energy release in muscle cells for retaining the level of activity. Therefore, the role of creatin in its uptake and recycle is significant in anerobic energy release in muscle tissues during strenuous exercise.

If a naturally derived safe composition can help in this segment, it enhances the endurance of athlete. There is an unmet need for such a composition. Therefore, there is an increasing demand for a safe and efficacious composition for improving anabolic activity while improving immunity and creatin uptake and recycles in muscle cells.

### OBJECTS OF THE PRESENT INVENTION

The principal object of the present invention is to obtain a novel composition comprising saponin and a sugar derivative.

Another object of the present invention is to obtain the novel composition comprising saponin and a sugar derivative from the Trigonella species.

Yet another object of the present invention is to develop a process for the preparation of the novel composition comprising saponin and a sugar derivative from Trigonella species.

Still another object of the present invention is to use the composition for improving the physiological factors.

Still another object of the present invention is to use the composition for increasing the levels of testosterone and lymphocytes.

### STATEMENT OF THE INVENTION

Accordingly, the present invention relates to a composition comprising saponin of structural formula I

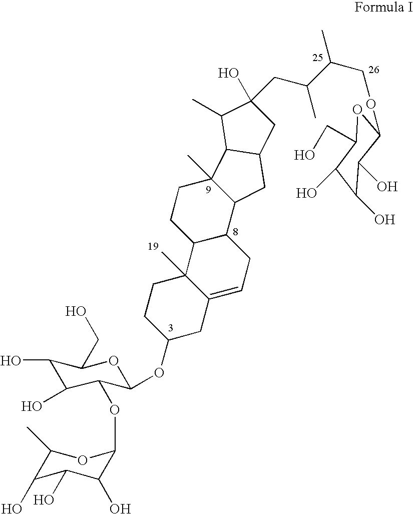

and sugar derivative of structural formula II

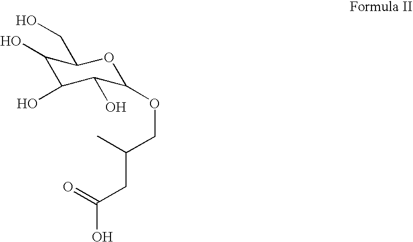

for improving physiology, optionally along with one or more pharmaceutically acceptable excipients; a process for preparation of a composition comprising saponin of structural formula I

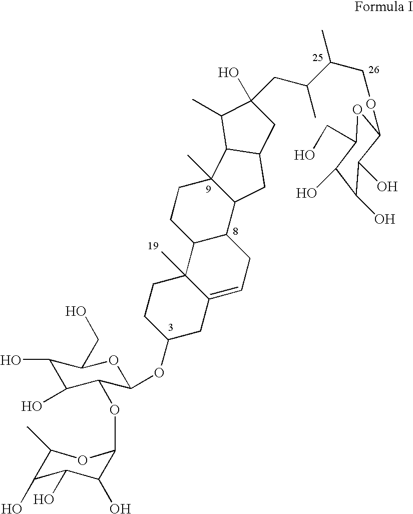

and sugar derivative of structural formula II

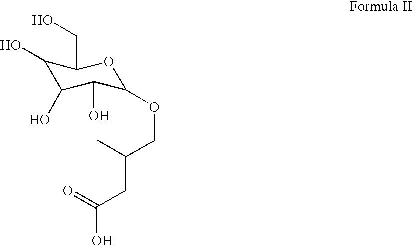

obtained from Trigonella. species for improving physiology, said process comprising steps of:
a. defatting flaked fenugreek seeds with n-hexane and hydro-alcohol to remove fatty matter;
b. concentrating the defatted extract under vacuum to obtain a mass;
c. dissolving concentrated mass to obtain a clear solution;
d. passing clear solution through ion-exchange resin and adsorbent column to retain active compounds; and
e. eluting adsorbed active compounds using alcohol followed by hydro-alcohol and concentrating both the fractions individually to a semi solid mass; and
f. redissolving the semi solid mass in an aqueous solvent followed by filtration and drying to obtain the composition and a method for improving physiology factors in a subject in need thereof, said method comprising step of administering pharmaceutically effective amount of composition comprising saponin of structural formula I

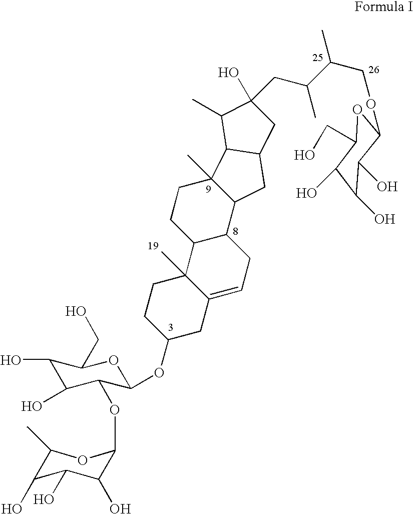

and sugar derivative of structural formula II, optionally along with one or more pharmaceutically acceptable excipients to the subject.

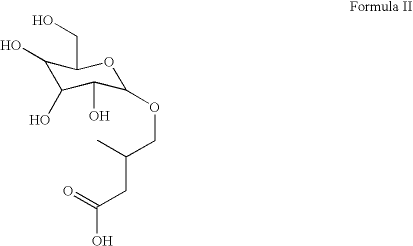

### DETAILED DESCRIPTION OF THE PRESENT INVENTION

The present invention relates to a composition comprising saponin of structural formula I

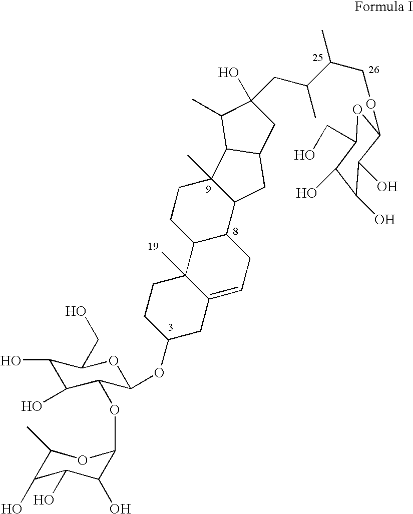

and sugar derivative of structural formula II

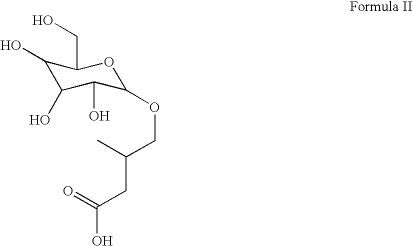

for improving physiology, optionally along with one or more pharmaceutically acceptable excipients.

In another embodiment of the present invention, said saponin and sugar derivative are obtained from plant Trigonella species, preferably Trigonella foenum graecum.

In yet another embodiment of the present invention, said plant parts are roots, shoots, leaves, seeds, entire plant and preferably seeds.

In still another embodiment of the present invention, said saponin is 26-0-β-D Glucopyranosyl pseudodiosgenin-3-0-∝-L-Rhamnopyranosyl 1-(1-2)-β-D Glucopyranoside; and the sugar derivative is γ-methyl δ-hydroxy Pentanoic acid β-glucopyranoside.

In still another embodiment of the present invention, said saponin and sugar derivative are mixed in propositions ranging from 5% to 80% of saponin, preferably 10% to 70% of saponin.

In still another embodiment of the present invention, said excipients(s) are selected from a group comprising granulating agents, binding agents, lubricating agents, disintegrating agents, sweetening agents, glidants, anti-adherents, anti-static agents, surfactants, anti-oxidants, gums, coating agents, coloring agents, flavouring agents, coating agents, plasticizers, preservatives, suspending agents, emulsifying agents and speheronization agents.

In still another embodiment of the present invention, said composition is formulated into various dosage forms selected from a group comprising tablet, troches, lozenges, aqueous or oily suspensions, ointment, patch, gel, lotion, dentifrice, capsule, emulsion, creams, spray, drops, dispersible powders or granules, emulsion in hard or soft gel capsules, syrups, elixirs, phytoceuticals, nutraceuticals and food stuffs.

In still another embodiment of the present invention, said composition is free of adverse effects.

The present invention also relates to a process for preparation of a composition comprising saponin of structural formula I

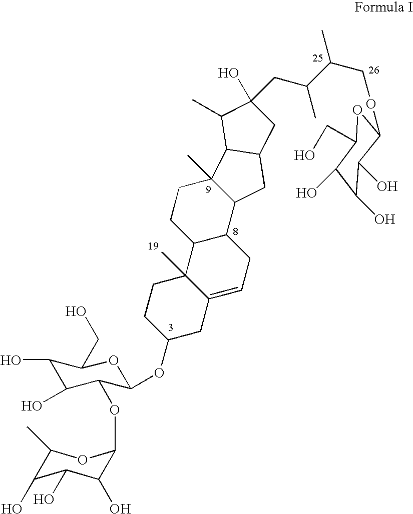

and sugar derivative of structural formula II

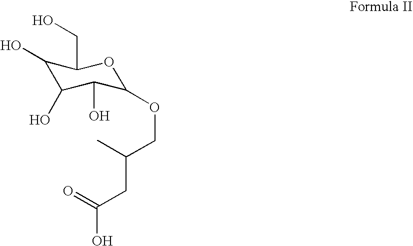

obtained from Trigonella species for improving physiology, said process comprising steps of:
a. defatting flaked fenugreek seeds with n-hexane and hydro-alcohol to remove fatty matter;
b. concentrating the defatted extract under vacuum to obtain a mass;
c. dissolving concentrated mass to obtain a clear solution;
d. passing clear solution through ion-exchange resin and adsorbent column to retain active compounds; and
e. eluting adsorbed active compounds using alcohol followed by hydro-alcohol and concentrating both the fractions individually to a semi solid mass; and
f. redissolving the semi solid mass in an aqueous solvent followed by filtration and drying to obtain the composition.

In another embodiment of the present invention, said defatting is carried out for a time period ranging from 8 to 12 hrs and preferably 10 hrs.

In yet another embodiment of the present invention, said defatting is carried out at a temperature ranging from 30 to 40° C. and preferably 35° C.

In still another embodiment of the present invention, the hydro alcohol for defatting is isopropyl alcohol and water are at a ratio of about 4:1.

In still another embodiment of the present invention, said extract is concentrated under vacuum at a temperature ranging from 45 to 55° C. and preferably 50° C.

In still another embodiment of the present invention, said mass is dissolved in deionized water to get clear solution.

In still another embodiment of the present invention, said ion-exchange resin is cation exchange gel type resin.

In still another embodiment of the present invention, said hydro alcohol for eluting adsorbed compounds is water and methanol at a ratio of 1:1.

The present invention is in relation to a method for improving physiology factors in a subject in need thereof, said method comprising step of administering pharmaceutically effective amount of composition comprising saponin of structural formula I

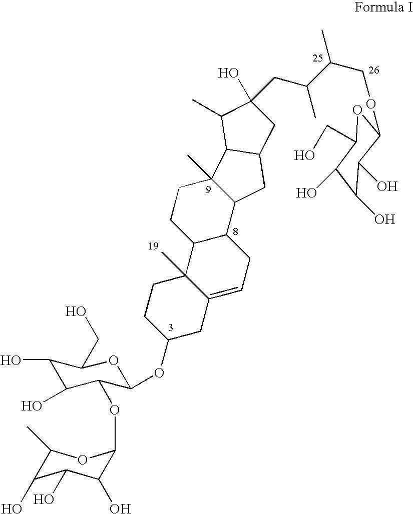

and sugar derivative of structural formula II, optionally along with one or more pharmaceutically acceptable excipients to the subject.

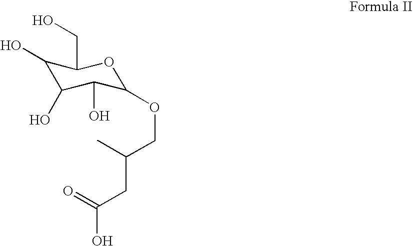

In another embodiment of the present invention, the subject is an animal or human being.

In yet another embodiment of the present invention, said physiology factor is related to the anabolic activity having an increase in testosterone, prolactin, creatinine uptake, nitrogen retention and immunity in the subject.

In still another embodiment of the present invention, said composition is administered at a dose of about 600 mg per day.

In still another embodiment of the present invention, said composition has shown increase in immunity.

In still another embodiment of the present invention, said composition on continued usage will not lead to increase in any World Anti Doping Agency prohibited compounds in serum.

One embodiment of the present invention relates to a novel herbal composition for improving physiology factors, wherein the composition comprises a saponin and a sugar derivative optionally along with one or more pharmaceutically acceptable additives. Also another key feature of this novel composition is the absence of literature reported Fenusides (saponins).

Another embodiment of the present invention also relates to a process for the preparation of novel herbal composition, wherein said composition comprises steps of flaking, defatting, solvent extraction of fenugreek seeds followed by concentrating the extract to obtain the saponin and sugar derivative. In one aspect of this embodiment, the present invention relates to the following process:
1) Fenugreek seeds are flaked to expose the inner core so as to ensure effective defatting, extraction and processing.
2) Hexane solvent is passed through the fenugreek bed repeatedly to achieve effective defatting of the fenugreek
3) The flaked fenugreek seeds are loaded in a percolator and solvent mixture comprising of an aliphatic alcohol and water are passed through the fenugreek layer to achieve effective extraction of the Saponin
4) The solvent is vacuum concentrated at lower temperatures to ensure the integrity of the Saponin and the resultant mass is dissolved in deionised water to get clear solution.
5) The clear solution thus obtained is passed through a polymeric adsorbent XAD-7 to retain the Saponin having high molecular weight.
6) The column is washed free of all impurities and colors using 0.1% sodium chloride solution and the adsorbed material is eluted with a solvent consisting of an alcohol and water in gradient manner. The alcohol used is methyl alcohol, ethyl alcohol, isopropyl alcohol for this purpose.
7) The eluted compound in the solvent elute is concentrated individually as the alcoholic fraction, which is a saponin and the water fraction which is a glucoside derivative under vacuum to remove the solvent and the resultant mass is dissolved and filtered to get a clear solution
8) The clear solution obtained is spray dried and reconstituted to get a free flowing powder of the following composition consisting of saponin ranging from 10% to 80% and the glucoside derivative remaining along with excipients. The structure and the analytical methods have been elucidated to maintain the integrity of the composition for the purpose of administration.

A third embodiment of the present invention also relates to the use of the novel herbal composition comprising saponin and sugar derivatives optionally along with one or more pharmaceutically acceptable additives to manufacture a medicament for improving physiology factors in subjects in need thereof. In one aspect of this embodiment, enhancing anabolic activity related factors includes increasing the weight of organs like seminal vesicle, prostate and levator Ani muscles. Another aspect of this embodiment includes improving the body composition of a subject in need thereof. Another aspect of the present embodiment is an increase in levels of prolactin in the subject. Another aspect of the present embodiment in an increase in lymphocytes. Another aspect of the present embodiment is enhancing immunity.

A fourth embodiment of the present invention includes the use of the novel herbal composition comprising saponin and sugar derivatives optionally along with one or more pharmaceutically acceptable additives to manufacture a medicament for enhancing testosterone levels in a subject in need thereof, One aspect of the present embodiment comprises promoting anabolic muscle building in subjects. Another aspect of the present embodiment is an enhancement in creatine reuptake and recycle.

One aspect of the process of extraction of the composition comprising saponins and sugar derivatives is further described below:

Fenugreek seeds were flaked using roller flaking machine to size of thickness varying between 1 mm to 3 mm size. The effective exposure of the inner core was achieved by flaking to a size preferably of 1 mm thickness. The flaked seeds were packed in an extractor fitted with bottom filter of suitable mesh size preferably 200 mesh so as not to allow the seed meal down along with solvent. Hexane is allowed to percolate through the packed fenugreek layer. The percolated solvent is recycled efficiently over period of 10 to 12 hrs so that the resultant Fenugreek meal is free of oils and lipids.

The Hexane extracted meal is re-extracted with a solvent mixture comprising of aqueous aliphatic alcohol in the aqueous to alcohol ratio of 1:1 to 9:1 preferably 1:7 as the solvent. The said alcohol may be methanol, Ethanol, Isopropanol and preferably ethanol as the alcoholic solvent. The aqueous alcohol mixture is passed from top to bottom through the fenugreek layer in the percolator. The process of recycling the solvent was continued for a period of time ranging between 3 hrs to 10 hrs preferably 8 hrs at room temperature. The clear extract from the bottom of the percolator is inspected visually for any suspended particles. And refiltered if necessary. The clear filtrate is vacuum concentrated at temperature ranging between 50° C. to 75° C. preferably at 55° C. to a pasty mass. The paste is re-dissolved in deionised water to a clear solution consisting of around 5% solid content.

The clear solution is passed through an adsorbent column consisting of polymer adsorbent amberlyt XAD-761. and eluted with a mixture of solvent comprising of an alcohol and water in the ratio of 1;1 The said alcohol is preferably Ethanol Isopropanol or methanol can also be used for the desorbtion cycle. The desorbed material solution is vacuum concentrated to 50% solid content and spray dried in a co current spray dryer to powder. The powder obtained by the above manner is tested on Swiss wistar rats for testosterone like activity, anabolic activity and androgenic activity by feeding them at dosage level ranging between 5 mg/kg to 50 mg/kg. In an embodiment of the present invention, where in the invention relates to process for obtaining fenugreek Saponin in the following manner.
1. Fenugreek seeds are flaked to expose the inner core so as to ensure effective defatting, extraction and processing.
2. Hexane solvent is passed through the fenugreek bed repeatedly to achieve effective defatting of the fenugreek
3. The flaked fenugreek seeds are loaded in a percolater and solvent mixture comprising of an aliphatic alcohol and water are passed through the fenugreek layer to achieve effective extraction of the Saponin
4. The solvent is vacuum concentrated at lower temperatures to ensure the integrity of the Saponin and the resultant mass is dissolved in deionised water to get clear solution.
5. The clear solution thus obtained is passed through a polymeric adsorbent XAD-7 to retain the Saponin having high molecular weight.
6. The column is washed free of all impurities and colours using 0.1% sodium chloride solution and the adsorbed material is eluted with a solvent consisting of an alcohol and water in gradient manner The alcohol used is methylalcohol, ethylalcohol, isopropylalcohol for this purpose.
7. The eluted compound in the solvent elute is concentrated individually as the alcoholic fraction, which is a saponin and the water fraction which is a glucoside derivative under vacuum to remove the solvent and the resultant mass is dissolved and filtered to get a clear solution
8. The clear solution obtained is spraydryed and reconstituted to get a free flowing powder. of the following composition consisting of saponin ranging from 10% to 80% and the glucoside derivative remaining along with excepients. The structure and the analytical methods have been elucidated to maintain the integrity of the composition for the purpose of administration.

The technology of the instant Application is further elaborated with the help of following examples. However, these examples should not be construed to limit the scope of the invention.

### EXAMPLE 1

1000 gms of fenugreek seeds having a moisture content less than 7% were flaked in a roller flaker to a thickness of 1 mm The flaked material is stalked in a column having abed height of 300 mm. 5 liters of hexane was passed through the layer of fenugreek and the elluent collected from the bottom is recycled through the fenugreek layer for a period of 10 hrs at 35° C. After 10 hrs the fenugreek layer was drained free of hexane. Solvent mixture (8 liters) comprising of Isopropyl alcohol and water in the ratio of 4:1 was passed through the layer for a period of 8 hrs at 35° C. by recycling the elluent. After 8 hrs the fenugreek bed is drained free of extracts and all the collected extracts. The collected extracts were concentrated to semisolid mass under vacuum at 50° C. The concentrated mass is re-dissolved in 5 liters of deionised water to get a clear solution. The clear aqueous solution was passed through a column containing 500 ml of XAD-761 slowly over a period of 2 hrs. The column elluents were checked by TLC screening for the presence of Saponin (TLC system chloroform, methanol and water in the ratio 65:33:2) detection using Ehrlich reagent.

After completion of the column the resin bed was washed free of colors and adhering impurities using 200 ml of 1% sodium chloride solution. The column is desorbed using 800 ml of methanol at rate of 400 ml per hour and the fractions are collected in 50 ml volumes and screened for the major saponin spot using above mentioned TLC technique After the completion of methanol fraction The solvent composition was changed in a gradient manner to a mixture of methanol water of total 3 bed volumes (1500 ml) the fraction eluted at the solvent ratio of 1:1 of methanol:water was pooled separately. The methanol fraction rich in saponin and the mixed solvent fraction rich in the sugar derivative are concentrated individually at 45° C. to a semisolid mass. The dried mass is re-dissolved in 150 ml of deionised water and filtered free of insoluble. The solution is concentrated under vacuum at 50° C. to a solid content of 20% and spray dried in a co-current indirect hot air spray dryer under following conditions.

Inlet temperature: 160° C.
Outlet temperature: 80° C.

### Atomizer RPM: 12000.

After carrying out HPLC analysis using the following method the saponin fraction and the sugar derivative fraction are mixed and reconstituted in various proposition ranging from 10% to 70% of saponin and remaining the glucoside derivative.

### EXAMPLE 2

The composition is tested and analyzed as per the following analytical procedure. Dissolve 1 gm. of the final spray dried material into Analytical grade IPA (Isopropyl alcohol) about 10 ml. Sonicate for 15 minutes. The saponin fraction dissolves into IPA readily. There is an insoluble fraction. Filter this into IPA soluble fraction and IPA insoluble fraction.

IPA solution fraction is vacuum distilled and made into a powder under vacuum tray drying. This powder is tested for saponin content by HPLC

HPLC METHOD

| | |
|---|---|
| Column: | RP C-18, 5 Micron size 150 × 4.6 mm |
| Flow rate | 1 ml/min |
| Detector wavelength: | 205 nm UV |

Mobile Phase

| Time | Water:Acetonitrile |
|---|---|
| 0 | 80:20 |
| 20 | 65:35 |

The saponin major appears at 3.5 mins. The saponin fraction is about 55-60% by HPLC.

The IPA insoluble fraction is washed twice with clean IPA and tray dried under vacuum. This is weighed. This is about 350 gms. Thus the IPA insoluble fraction which is Methyl Hydroxy Pentanoic Acid Glucopyranoside is about 35%.

### EXAMPLE 3

1000 gms of fenugreek seeds having a moisture content less than 7% were flaked in a roller flaker to a thickness of 1 mm The flaked material is stalked in a column having abed height of 300 mm. 5 liters of hexane was passed through the layer of fenugreek and the elluent collected from the bottom is recycled through the fenugreek layer for a period of 10 hrs at 35° C. After 10 hrs the fenugreek layer was drained free of hexane. Solvent mixture (8 liters) comprising of Ethyl alcohol and water in the ratio of 4:1 was passed through the layer for a period of 8 hrs at 35° C. by recycling the elluent. After 8 hrs the fenugreek bed is drained free of extracts and all the collected extracts. The collected extracts were concentrated to semisolid mass under vacuum at 50° C. The concentrated mass is re-dissolved in 5 liters of demonized water to get a clear solution. The clear aqueous solution was passed through a column containing 500 ml of XAD-761 slowly over a period of 2 hours the column elluent were checked by TLC screening for the presence of Saponin. (TLC system chloroform, methanol and water in the ratio 65:33:2) detection using Ehrlich reagent. After completion of the column the resin bed was washed free of colures and adhering impurities using 200 ml of 1% sodium chloride solution. The column is desorbed using 800 ml of methanol at rate of 400 ml per hour and the fractions are collected in 50 ml volumes and screened for the major saponin spot using above mentioned TLC technique

After the completion of methanol fraction the solvent composition was changed in a gradient manner to a mixture of methanol water of total 3 bed volumes (1500 ml). The fraction eluted at the solvent ratio of 1:1 of methanol: water was pooled separately. The methanol fraction rich in saponin and the mixed solvent fraction rich in the sugar derivative are concentrated individually at 45° C. to a semisolid mass. The dried mass is re-dissolved in 150 ml of deionised water and filtered free of insoluble. The solution is concentrated under vacuum at 50° C. to a solid content of 20% and spray dried in a co-current indirect hot air spray dryer under following conditions.

Inlet temperature: 160° C.
Outlet temperature: 80° C.

### Atomizer RPM: 12000.

After carrying out HPLC analysis using the following method the saponin fraction and the sugar derivative fraction are mixed and reconstituted in various proposition ranging from 10% to 70% of saponin and remaining the glucoside derivative and excipients

### EXAMPLE 4

1000 gins of fenugreek seeds having a moisture content less than 7% were flaked in a roller flaker to a thickness of 1 mm The flaked material is stalked in a column having abed height of 300 mm. 5 liters of hexane was passed through the layer of fenugreek and the elluent collected from the bottom is recycled through the fenugreek layer for a period of 10 hrs at 35° C. After 10 hrs the fenugreek layer was drained free of hexane. Solvent mixture (8 liters) comprising of Methanol and water in the ratio of 4:1 was passed through the layer for a period of 8 hrs at 35° C. by recycling the elluent. After 8 hrs the fenugreek bed is drained free of extracts and all the collected extracts. The collected extracts were concentrated to semisolid mass under vacuum at 50° C. The concentrated mass is re-dissolved in 5 liters of demonized water to get a clear solution. The clear aqueous solution was passed through a column containing 500 ml of XAD-761 slowly over a period of 2 hours the column elluent were checked by TLC screening for the presence of Saponin. (TLC system chloroform, methanol and water in the ratio 65:33:2) detection using Ehrlich reagent.

After completion of the column the resin bed was washed free of colures and adhering impurities using 200 ml of 1% sodium chloride solution. The column is desorbed using 800 ml of methanol at rate of 400 ml per hour and the fractions are collected in 50 ml volumes and screened for the major saponin spot using above mentioned TLC technique After the completion of methanol fraction The solvent composition was changed in a gradient manner to a mixture of methanol water of total 3 bed volumes (1500 ml). The fraction eluted at the solvent ratio of 1:1 of methanol: water was pooled separately. The methanol fraction rich in saponin and the mixed solvent fraction rich in the sugar derivative are concentrated individually at 45° C. to a semisolid mass. The dried mass is re-dissolved in 150 ml of deionised water and filtered free of insoluble. The solution is concentrated under vacuum at 50° C. to a solid content of 20% and spray dried in a co-current indirect hot air spray dryer under following conditions.

Inlet temperature: 160° C.
Outlet temperature: 80° C.

### Atomizer RPM: 12000.

After carrying out HPLC analysis using the following method the saponin fraction and the sugar derivative fraction are mixed and reconstituted in various proposition ranging from 10% to 70% of saponin and remaining the glucoside derivative.

### EXAMPLE 5

The individual fractions thus collected and screened for the major saponin was further crystallized in methanol to get a crystalline material the 13-C NMR DATA (in pyridine D-6) is as follows corresponding to the Furastanol glycoside of the following

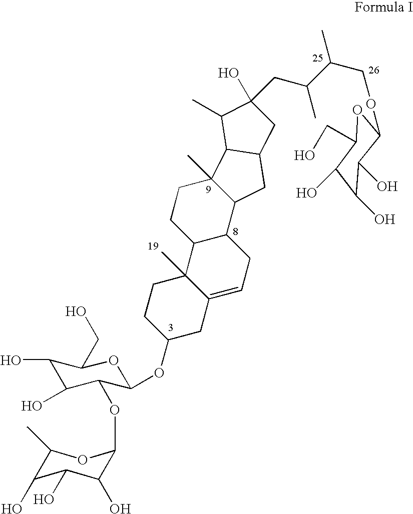

The saponin is identified as 26-0-β-D Glucopyranosyl pseudodiosgenin-3-0-∝-L-Rhamnopyranosyl 1-(1-2)-β-D Glucopyranoside.

### Molecular Formula C₄₇H₇₈O₁₇

Molecular weight 914

| | |
|---|---|
| AGLYCON | GLUCON |

| NO | PPM | NO | PPM |
|---|---|---|---|
| 1 | 37.5 | 26-0-GLU | |
| 2 | 30 | 1 | 104.9 |
| 3 | 78 | 2 | 75 |
| 4 | 39 | 3 | 72.5 |
| 5 | 140 | 4 | 71.5 |
| 6 | 121 | 5 | 78 |
| 7 | 32 | 6 | 62.5 |
| 8 | 31 | 2-RHA | |
| 9 | 50 | 1 | 101 |
| 10 | 37 | 2 | 72.5 |
| 11 | 21 | 3 | 76.5 |
| 12 | 40 | 4 | 74 |
| 13 | 40.5 | 5 | 70 |
| 14 | 56.5 | 6 | 19 |
| 15 | 31.5 | 3-0-glu | |
| 16 | 81 | 1 | 100 |
| 17 | 64 | 2 | 78.5 |
| 18 | 16 | 3 | 76 |
| 19 | 19 | 4 | 78 |
| 20 | 40.5 | 5 | 77.5 |
| 21 | 16.5 | 6 | 61.5 |
| 22 | 110 | | |
| 23 | 31 | | |
| 24 | 28 | | |
| 25 | 34 | | |
| 26 | 75 | | |
| 27 | 17.5 | | |

### EXAMPLE 6

The water soluble fraction named as the sugar derivative was further cleaned up with charcoal treatment followed by concentration under vacuum at 40° C. and isolated by alcohol precipitation was dried. Structure elucidation using proton as well as 13C NMR was carried out and it was identified as γ-methyl δ-hydroxy Pentanoic acid β-glucopyranoside.

### Molecular Formula C₁₁H₂₀O₈

Molecular weight 280
NMR Confirms the following structure

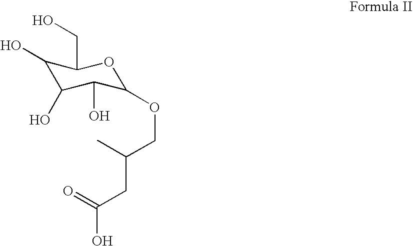

γ-methyl δ-hydroxy Pentanoic acid β-glucopyranoside

13 C NMR

| No | Ppm |
|---|---|
| 1 | 104 |
| 2 | 74 |
| 3 | 73 |
| 4 | 71 |
| 5 | 77 |
| 6 | 62 |
| 7 | 75 |
| 8 | 31 |
| 9 | 40 |
| 10 | 125 |
| 11 | 17 |
| 12 | |
| 13 | |

### EXAMPLE 7

### Anabolic Activity

Male Adolescent rats of Swiss Wistar strain of body weight 55 gms are chosen for this experiment and are castrated. The castrated groups are divided into 2 groups.
1. Control group castrated animals n=6
2. Drug treated castrated animals treated with test compound at 25 mg/kg n=6

Both the groups are fed ad libitum food and water and observed for 28 days. Their serum at the beginning of the experiment and at the end of the experiment are drawn and checked for Total testosterone, and free testosterone. The animals are sacrificed at the end of the experiment and seminal vesicle; ventral Prostate and Levator Ani are removed and weighed. These organ weights are compound with the control animals.

### Anabolic Activity: Organ Weight

| | Organ weight (g) | | |
|---|---|---|---|
| | Seminal | | Levator |
| Group | vesicle | Prostate | ANI |
| Castrated Control Group | 2.58 | 4.52 | 39.35 |
| Castrated Test compound | 5.70 | 20.74 | 53.23 |
| Group | | | |
| Pure Saponin | 2.36 | 5.3 | 36.7 |

From the above data we see that there is an increase in organ weight in the test group. Also seen is that the specified pure saponin isolated from Fenugreek does not show anabolic activity in castrated animals. Similar observation was noticed when the saponin of instant invention alone tested. But, when the saponin of instant invention combined with the sugar derivative of instant invention the anabolic activity was found to be enhanced or extraordinary anabolic activity vis-à-vis the pure conventional saponin.

### Testosterone

| | SERUM TOTAL | | SERUM FREE | |
|---|---|---|---|---|
| | TESTOSTERONE | | TESTOSTERONE | |
| | (ng/ml) | | (Pg/ml) | |
| | Before | After | Before | After |
| GROUP | Castration | Castration | Castration | Castration |
| Control | 51.5 | 23 | 1.53 | 0.55 |
| Test | 54.3 | 21 | 1.61 | 0.82 |
| Compound | | | | |

The above experiments confirm the activity of the test compound as one that enhances anabolic activity. In this experiment, of particular interest is the increase in weight of the Levitor Ani muscles. This increase in weight is an indication of the potential muscle building properties of this compound. Also anabolic activity is mediated by androgenic hormones like testosterone. Hence an increase in testosterone levels is an indication of anabolic activity.

### Conclusion:

In summary, it was concluded that the saponin of structural formula I of instant invention when tried alone does not showed any activity. On the contrary the same when combined with the sugar derivative of structural formula II of instant invention showed enhanced anabolic activity. Thus, the composition of instant invention is not only novel but also inventive enough as its activity is enhanced or extraordinary vis-à-vis the pure conventional saponins.

### EXAMPLE 8

### Human Study

A prospective Double Blind Randomized Placebo controlled study in 60 homogenous subjects. Subjects performed resistance training minimum one hour per day for 5 days a week, for 8 weeks under the supervision of trained supervisors and a sports medicine specialist. The test compound was administered at a dose of given 600 mg per day in a single dose.

| Group | Mean Age | Mean Height | Mean Weight |
|---|---|---|---|
| Test Compound | 23.21 | 165.52 | 64.93 |
| Placebo | 21.62 | 168.35 | 64.90 |

### Observations:

Test Compound group showed an increase in muscle mass by decrease in skin fold thickness of Triceps and Thighs.

Percentage change with
respect to Base line

| Body Composition | Test Compound | Placebo |
|---|---|---|
| Triceps | 14.52 Decrease | 5.79 Decrease |
| Thighs | 12.66 Decrease | 6.45 Decrease |
| Chest | 19.52 Decrease | 23.55 Decrease |
| Weight | 0.03 Increase | 1.48 Increase |

### Biochemical Parameters

The test compound shows an increase in anabolic activity characterized by muscle building capacity of the subject. Also the test compound increased nitrogen uptake which is essential for muscle building. This nitrogen uptake is shown by a reduction in the blood urea nitrogen (BUN) levels

Percentage change
with respect to Baseline

| Parameter | Test Compound | Placebo |
|---|---|---|
| BUN | 7.92 Decrease | 1.36 Increase |

### P<0.05

The difference with respect to placebo is significant. This confirms Anabolic Activity increase in the subjects taking the test composition.

### Free Testosterone

Percentage change
with respect to Baseline

| Parameter | Test Compound | Placebo |
|---|---|---|
| Free Testosterone | 96% Increase | 48% Increase |

### P<0.003

There is a 96% increase in free testosterone levels. This is a very important characteristic. In the serum almost 98% of the testosterone present is in the bound form. This form is inactive. Only free testosterone contributes to anabolic activity. Also another important feature of the test compound is the increase in free testosterone while maintaining the serum testosterone within physiological limits. Free testosterone in Test Compound after 8 weeks at 35.29 pg/ml. (Male: 8.69 to 54.69)

As per the Sports medicine Vol. 26, number 2, August 1998, pp 101-117 Kargotich, Goodman Univ: west Australia
The influence of exercise induced plasma volume changes on the interpretation of Biochemical Parameters
This explains Placebo increase.
Adjusting this plasma volume induced increase in free Testosterone, Test Compound has shown 48% increase in free Testosterone. P<0.003. The test compound increases Physiologically active testosterone in serum.

### Serum Total Testosterone

Percentage change
with respect to Baseline

| Parameter | Test Compound | Placebo |
|---|---|---|
| Serum Total Testosterone | 11.3 Increase | 13.9 Increase |

No significant increase in each group after plasma volume correction.

### Immunity

Test Compound not only compensates the impairment of immunity but also stimulates it by 11.21%

Percentage change
with respect to Baseline

| Parameter | Test Compound | Placebo |
|---|---|---|
| Lymphocytes | 11.21% Increase | 7.29% Decrease |

### P<0.003

The test compound has increased immunity after intense exercise regimen compound to placebo significantly.

### Serum Creatinine Levels

Test Compound recycles Creatine in muscle cells with minimal degradation

Percentage change
with respect to Baseline

| Parameter | Test Compound | Placebo |
|---|---|---|
| Serum Creatinine | 17.5% Decrease | 8.5% Decrease |

### P<0.05

Test compound helps in creative uptake and recycle in muscle cells. This releases energy anerobically.

### DHEA and DHEA Sulphate

Test compound and Placebo group had same level of DHEA and DHEA Sulphate after 8 weeks

| | Serum levels after 8 weeks | | |
|---|---|---|---|
| Parameter | Test Compound | Placebo | Physiological limits |
| DHEA ng/ml | 5.25 | 5.62 | 1.8-12.5 |
| DHEA Sulphate μg/ml | 192.50 | 215.50 | 280-640 |

Hypothesis in Tribulus Terrestris—Saponins are precursors of DHEA and their action is mediated by DHEA formation.

This has been tested and found that the action of the test compound is not mediated by DHEA and DHEA Sulphate. This also helps to confirm the fact that the continued use of test compound will not lead to increase in any WADA (World Anti Doping Agency) prohibited compound in serum.

### Prolactin

Test compound increased prolactin significantly compared to placebo.

Percentage change
with respect to Baseline

| Parameter | Test Compound | Placebo |
|---|---|---|
| Prolactin | 70% Increase | 20% Increase |

### P<0.003

Prolactin levels after treatment with the Test Compound group is 10.95 ng/ml. This is within physiological limits (Males 2.1 to 17.7)

Increase in Prolactin Incidcates
1. Exercise (stress) in both groups.
2. High free Testosterone gets degraded by Aromatase to Estradiol. Estradiol causes Prolactin secretion. This confirms increase in Free Testosterone in Test compound group.
3. Lymphocytes Secrete Prolactin. Test compound group has increased Lymphocytes.

The above description is illustrative of the various embodiments of the invention and is not to be construed as limiting, it being understood that a person skilled in the art may carry out many obvious variations to the present invention.

We claim:

1) A composition comprising saponin of structural formula I 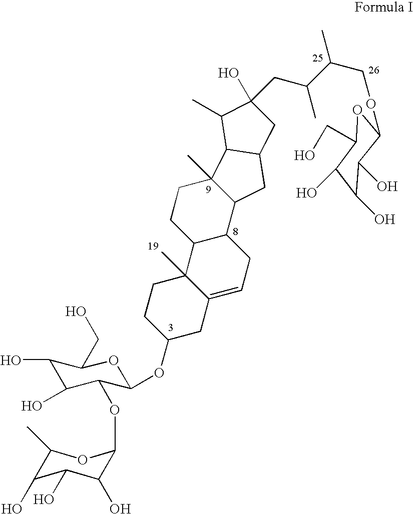
and sugar derivative of structural formula II 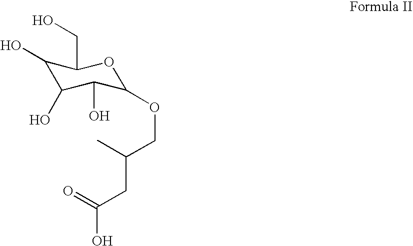
for improving physiology, optionally along with one or more pharmaceutically acceptable excipients.

2) The composition as claimed in claim 1, wherein said saponin and sugar derivative are obtained from plant Trigonella species, preferably Trigonella foenum graecum.

3) The composition as claimed in claim 2, wherein said plant parts are roots, shoots, leaves, seeds, entire plant and preferably seeds.

4) The composition as claimed in claim 1, wherein said saponin is 26-0-β-D Glucopyranosyl pseudodiosgenin-3-0-∝-L-Rhamnopyranosyl 1-(1-2)-β-D Glucopyranoside; and the sugar derivative is γ-methyl δ-hydroxy Pentanoic acid β-glucopyranoside.

5) The composition as claimed in claim 1, wherein said saponin and sugar derivative are mixed in propositions ranging from 5% to 80% of saponin, preferably 10% to 70% of saponin.

6) The composition as claimed in claim 1, wherein said excipients(s) are selected from a group comprising granulating agents, binding agents, lubricating agents, disintegrating agents, sweetening agents, glidants, anti-adherents, anti-static agents, surfactants, anti-oxidants, gums, coating agents, coloring agents, flavouring agents, coating agents, plasticizers, preservatives, suspending agents, emulsifying agents and speheronization agents.

7) The composition as claimed in claim 1, wherein said composition is formulated into various dosage forms selected from a group comprising tablet, troches, lozenges, aqueous or oily suspensions, ointment, patch, gel, lotion, dentifrice, capsule, emulsion, creams, spray, drops, dispersible powders or granules, emulsion in hard or soft gel capsules, syrups, elixirs, phytoceuticals, nutraceuticals and food stuffs.

8) The composition as claimed in claim 1, wherein said composition is free of adverse effects.

9) A process for preparation of a composition comprising saponin of structural formula I 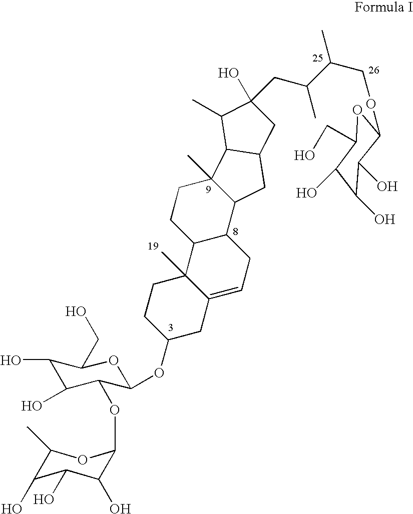
and sugar derivative of structural formula II 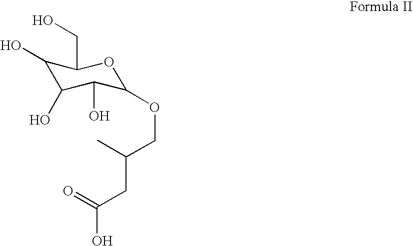
Obtained from Trigonella species for improving physiology, said process comprising steps of:
a. defatting flaked fenugreek seeds with n-hexane and hydro-alcohol to remove fatty matter;
b. concentrating the defatted extract under vacuum to obtain a mass;
c. dissolving concentrated mass to obtain a clear solution;
d. passing clear solution through ion-exchange resin and adsorbent column to retain active compounds; and
e. eluting adsorbed active compounds using alcohol followed by hydro-alcohol and concentrating both the fractions individually to a semi solid mass; and
f. redissolving the semi solid mass in an aqueous solvent followed by filtration and drying to obtain the composition.

10) The process as claimed in claim 9, wherein said defatting is carried out for a time period ranging from 8 to 12 hrs and preferably 10 hrs.

11) The process as claimed in claim 9, wherein said defatting is carried out at a temperature ranging from 30 to 40° C. and preferably 35° C.

12) The process as claimed in claim 9, wherein the hydro alcohol for defatting is isopropyl alcohol and water are at a ratio of about 4:1.

13) The process as claimed in claim 9, wherein said extract is concentrated under vacuum at a temperature ranging from 45 to 55° C. and preferably 50° C.

14) The process as claimed in claim 9, wherein said mass is dissolved in deionized water to get clear solution.

15) The process as claimed in claim 9, wherein said ion-exchange resin is cation exchange gel type resin.

16) The process as claimed in claim 9, wherein said hydro alcohol for eluting adsorbed compounds is water and methanol at a ratio of 1:1.

17) A method for improving physiology factors in a subject in need thereof, said method comprising step of administering pharmaceutically effective amount of composition comprising saponin of structural formula I 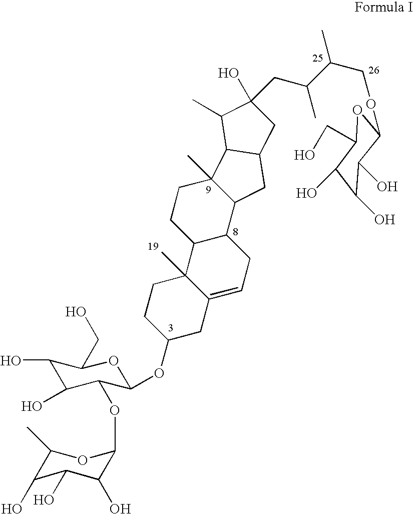
and sugar derivative of structural formula II, optionally along with one or more pharmaceutically acceptable excipients to the subject.

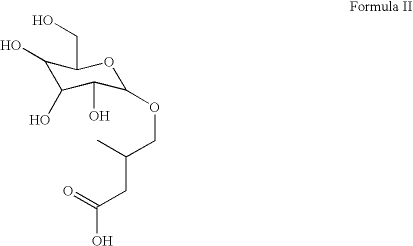

18) The method as claimed in claim 17, wherein the subject is an animal or human being.

19) The method as claimed in claim 17, wherein said physiology factor is related to the anabolic activity having an increase in testosterone, prolactin, creatinine uptake, nitrogen retention and immunity in the subject.

20) The method as claim in claim 17, wherein said composition is administered at a dose of about 600 mg per day.

21) The method as claimed in claim 17, wherein said composition has shown increase in immunity.

22) The method as claimed in claim 17, wherein said composition on continued usage will not lead to increase in any World Anti Doping Agency prohibited compounds in serum.
